(19) United States
(12) Patent Application Publication
(10) Pub. No.: US 2026/0263677 A1
(43) Pub. Date: Sep. 10, 2026

(54) REGULATION OF THE BODY TEMPERATURE OF A PATIENT

(71) Applicant: Maquet Cardiopulmonary GmbH, Rastatt (DE)

(72) Inventors: Peter van den OEVER, Best (NL); Peter RUTGERS, Enschede (NL); Ruben VERSCHOOF, Enschede (NL); Wessel SPEK, Hengelo (NL)

(21) Appl. No.: 19/665,886

(22) Filed: May 1, 2026

Related U.S. Application Data
(63) Continuation of application No. PCT/EP2024/081591, filed on Nov. 7, 2024.

(30) Foreign Application Priority Data
Nov. 10, 2023 (SE) 2351294-0

(51) Int. Cl. A61M 1/36; A61M 1/16; A61M 60/109; F25B 13/00

(52) U.S. Cl. CPC A61M 1/369; A61M 1/1698; A61M 1/367; A61M 60/109; F25B 13/00; A61M 2202/0413; A61M 2205/366; F25B 2300/00; F25B 2400/04

(57) ABSTRACT

A system for regulating a body temperature of a patient undergoing surgery is disclosed. The system comprises an extracorporeal circuit, a storage device for accumulating thermal energy, and a heat transfer system for transferring heat between the extracorporeal circuit and the storage device. The heat transfer system comprises a heat transfer circuit for circulating a thermal fluid, a heat exchanger for transferring heat between the extracorporeal circuit and the thermal fluid, and a heat pump circuit for transferring heat between the thermal fluid storage device. A method for regulating the body temperature is also disclosed.

### CROSS-REFERENCE TO RELATED APPLICATIONS

This application is a continuation under 35 U.S.C. § 120 of International Application No. PCT/EP2024/081591, filed Nov. 7, 2024, which is claims priority to SE Application No. SE 2351294-0, filed Nov. 10, 2023, under 35 U.S.C. § 119(a). Each of the above-referenced patent applications is incorporated by reference in its entirety.

### TECHNICAL FIELD

The present invention relates to body temperature regulation of patients undergoing surgery, and more specifically to systems and methods in which heat is transferred between blood in an extracorporeal circuit and a storage device.

### BACKGROUND

Hypothermia may be deliberately induced in a patient to reduce incidence of tissue damage during, for example, complex cardiac and vascular surgeries. Hypothermic circulatory arrest (HCA) and deep hypothermic circulatory arrest (DHCA) are examples of such techniques, which typically involve a systematic lowering of the patient's body temperature using a heater-cooler unit (HCU) during extracorporeal circulation of the blood. The HCU comprises a heat exchanger for transferring the heat from the blood to a water circuit. The heat may then be dissipated into the surrounding environment using an air-cooled heat exchanger.

Generally, it is desirable to lower the body temperature of the patient as fast as possible to reduce the total surgical time and hence the risk for medical complications. However, it may be challenging to efficiently dissipate large amounts of thermal energy in a short time while maintaining a controlled and sterile environment in the operating theatre. There is therefore a need for improved and alternative technologies for controlling the body temperature of a patient undergoing surgery.

### SUMMARY

In view of the above, it is thus an object of the present invention to provide an improved or alternative technology having the features set out in the independent claims.

Hence, according to a first aspect of the present invention, there is provided a system for regulating a body temperature of a patient undergoing surgery, comprising an extracorporeal circuit configured to circulate blood of the patient outside a body of the patient, a storage device configured to accumulate thermal energy, and a heat transfer system configured to transfer heat between the blood in the extracorporeal circuit and the storage device. The heat transfer system comprises a heat transfer circuit configured to circulate a thermal fluid, a first heat exchanger configured to transfer heat between the blood in the extracorporeal circuit and the thermal fluid, and a heat pump circuit configured to transfer heat between the thermal fluid and the storage device.

Maintaining a controlled and sterile environment in the operating theatre is crucial for the success of the surgical procedure and the prevention of postoperative complications, such as infections. The operating theatre often employs HEPA (High Efficiency Particulate Air) filters and a laminar airflow system to filter out contaminants from the air. The laminar airflow system typically delivers a unidirectional flow of air to “wash” away contaminants from the surgical field and reduce the risk for cross-contamination from other parts of the room. The laminar airflow can be contrasted with a turbulent airflow, which can mix clean and contaminated air and potentially carry airborne pathogens to the surgical field.

The use of air-cooled machinery in the operating theatre could pose challenges, as they risk circulating air that could potentially stir up dust or other particulates, disrupt the laminar airflow, and impact the temperature in the operating theatre. The need to maintain a laminar airflow and a controlled and sterile environment in the operating theatre therefore puts constraints on the rate with which heat can be dissipated from, for example, a heater-cooler unit (HCU).

The present invention makes use of the realisation that by transferring the heat to a storage device instead of dissipating the heat directly into the surrounding air, the impact on the air quality in the operating theatre can be reduced. By cooling the heat pump circuit by means of the storage device, a major part of the heat can be removed from the extracorporeal circuit and stored in the storage device without causing turbulence and spreading pathogens in the operating theatre.

Additionally, the storage device may act as a heat reservoir from which the stored heat can be transferred back to the patient to restore the body temperature after completion of the surgery.

The term “extracorporeal circuit” typically refers to a medical setup in which blood is circulated outside of the patient's body through a series of tubes, filters, and other medical devices for various therapeutic or diagnostic purposes. The circuit may comprise a fluid pump unit moving the blood through the circuit, and an oxygenator to oxygenate the blood and remove carbon dioxide. The extracorporeal circuit may, for example, perform the function of the heart and lungs during certain cardiac surgeries in which the heart needs to be stopped temporarily, and in critical care settings such as Extracorporeal Membrane Oxygenation (ECMO) to provide prolonged cardiac and respiratory support to patients whose heart and lungs are unable to provide an adequate amount of gas exchange.

The storage device, or thermal storage device, may be capable of storing heat by raising the temperature of a medium, such as a solid or a liquid (i.e., sensible heat storage), and/or by utilising phase-change materials (latent heat storage). The storage device may act as a reservoir capable of absorbing and releasing heat upon demand. Using a storage device is advantageous over air-cooled heat exchangers, as the latter tend to have a larger impact on the environment in the operating theatre.

By “heat pump” is generally understood an arrangement that transfers thermal energy from one area to another, such as from a colder area to a hotter area or from a hotter area to a colder area. Typically, the heat pump utilises a refrigeration cycle involving a compressor, an evaporator, a condenser, and an expansion valve to move heat against its natural flow direction (i.e., from cooler to hotter areas). The heat pump may also comprise a reverse valve arrangement for reversing the cycle, allowing heat to be moved from hotter to cooler areas. In the context of the present disclosure, the heat pump may be selectively operable in a cooling mode in which heat is transferred from the thermal fluid in the heat transfer circuit to the storage device, and in a heating mode in which heat is transferred from the storage device to the thermal fluid.

The heat pump can be contrasted with the heat exchanger, which may be understood as a component relying on the natural flow of heat from a hotter medium to a cooler medium. In that sense, the heat exchanger does not actively “pump” heat and does therefore not require external energy for the heat transfer itself.

The heat transfer system is arranged to transfer heat between the blood in the extracorporeal circuit and the storage device. For this purpose, the heat transfer system comprises a heat transfer circuit for circulating a thermal fluid carrying the heat. The heat transfer circuit may typically be a water circuit, in which the water forms the thermal fluid. The first heat exchanger is provided to transfer the heat between the blood and the thermal fluid without mixing the two, and the heat pump circuit is operable to transfer the heat between the thermal fluid and the storage device. Beneficially, the heat pump circuit allows for cooling of the blood to temperatures below the temperature of the storage device. The heat transfer system may thus be seen as a controllable thermal link between blood and the storage device.

In addition to the above, the heat transfer system may comprise a second heat exchanger for transferring the heat between the thermal fluid and the storage device. The heat may hence be transferred between the patient and the storage device in at least two different ways: by using the second heat exchanger, and by using the heat pump. These two different ways may be referred to as operating modes, in which the second heat exchanger represents a direct transfer mode, and the heat pump circuit represents an indirect transfer mode. A valve arrangement may be employed to control which one of heat pump circuit and the second heat exchanger to use, or if a combination of both should be used for the transfer of heat, for example during a transition mode. The valve arrangement may hence be configured to allow the thermal fluid to at least partly bypass at least one of the heat pump circuit and the second heat exchanger.

The choice of operating mode may be based at least in part on the difference between the patient's body temperature and the temperature of the storage device, as well as on the desired rate at which heat should is removed from the blood. The direct transfer mode, in which heat is transferred by the second heat exchanger, may be used in an initial cooling phase when the body temperature is relatively high, and the temperature of the storage device is relatively low. The relatively large temperature difference allows for an efficient operation of the second heat exchanger hence a relatively fast temperature reduction.

In a subsequent phase, when the difference in temperature of the body and the storage device has decreased (due to cooling of the patient and heating of the storage device) the indirect transfer mode may be initiated, in which the heat pump circuit is responsible for the heat transfer. As the heat pump circuit is capable of moving heat from a cooler area to a hotter area, the heat pump circuit can be operated to heat the storage device to temperatures exceeding the body temperature of the patient.

After the surgical procedure, when it is time to rewarm the patient, the system can initially be operated in the direct transfer mode in which the second heat exchanger allows heat to flow from the storage device to the patient. When the difference in temperature between the storage device is too small to allow efficient operation of the heat exchanger, the system may switch to the indirect transfer mode in which the heat pump circuit is operated in reversed mode to assist in the transfer of heat from the storage device to the patient.

In some embodiments, the heat transfer system comprises a heater for heating the thermal fluid in the heat transfer circuit. The heater may be arranged to provide additional heat, should the heat in the storage device not be sufficient to warm the patient to the desired temperature, or to increase the heat transfer rate during the rewarming phase.

In some embodiments, the heat pump circuit may comprise a supplementary, optional air-cooled heat exchanger for cooling a refrigerant of the heat pump circuit. The air-cooled heat exchanger may be used as a compliment to the storage device, for example to help shaving off peak loads or to assist in maintaining a stable body temperature during the surgery. Put differently, the air-cooled heat exchanger may be used in a steady-state phase during hypothermia to dissipate heat generated by the body and equipment used in the surgery. As a major part of the heat is, or has been, absorbed by the storage device, the air-cooled heat exchanger can be operated at a relatively low airflow with low risk of disturbing any laminar airflow. Beneficially, the additional cooling provided by the air-cooled heat exchanger may help reducing the size of the storage device.

In some embodiments, the storage device comprises a liquid, such as water, for accumulating thermal energy. The storage device may thus be a liquid reservoir, such as a container or tank, arranged to hold a certain volume of for example water. In an example, the storage device is referred to as a water tank.

In further examples, the storage device comprises a phase change material configured to absorb or release heat to/from the thermal liquid in the heat transfer circuit and/or the refrigerant of the heat pump circuit. The phase change material may for example be ice. The phase change material may be used instead of a liquid as described above, or in addition to such a liquid to further increase the heat storage capacity. The storage device may hence be a purely sensible heat storage device, a purely latent heat storage device, or a hybrid device combining the operation principles of a sensible heat storage and a latent heat storage. In some examples, the latent heat storage capacity is provided in form of ice arranged in, for example, a flexible container immersed in the water tank.

According to a second aspect, one or more non-transitory computer-readable media storing instructions executable by one or more processors is provided. The instructions, when executed, cause the one or more processors to perform operations comprising operating a first pump unit to circulate blood of the patient in the extracorporeal circuit, operating a second pump unit to circulate the thermal fluid in the heat transfer circuit, wherein the blood in the extracorporeal circuit and the thermal fluid in the heat transfer circuit are in thermal contact with each other to allow transfer of heat between the blood and the thermal fluid, and operating the heat transfer system to transfer heat between the thermal fluid in the heat transfer circuit and the storage device.

In some embodiments, the operations comprise at least one of operating a heat exchanger to transfer the heat between the thermal fluid and the storage device, and operating a heat pump circuit to transfer the heat between the thermal fluid and the storage device.

In some embodiments, the operations comprise:
determining a temperature difference between a body temperature of the patient and a temperature of the storage device;
comparing the temperature difference with a predetermined value;
operating the heat exchanger to transfer the heat from the thermal fluid to the storage device based at least in part on the temperature difference meeting or exceeding the threshold value; and
operating the heat pump circuit to transfer the heat from the thermal fluid to the storage device based at least in part on the temperature difference being below the threshold value.

In some embodiments, the operations comprise:
determining a temperature difference between the refrigerant of the heat pump circuit and a temperature of the storage device;
comparing the temperature difference with a predetermined threshold value; and
operating the air-cooled heat exchanger to cool the refrigerant based at least in part on the temperature difference being below the threshold value.

According to a third aspect of the present invention, a method for regulating the body temperature of a patient undergoing surgery is provided. The method comprises circulating blood of the patient in the extracorporeal circuit, circulating a thermal fluid in the heat transfer circuit, transferring heat between the blood in the extracorporeal circuit and the thermal fluid, and transferring heat between the thermal fluid and the storage device.

The second and third aspects may generally have the same features and advantages as the first aspect. It is further noted that the disclosure relates to all possible combinations of features, unless explicitly stated otherwise.

### BRIEF DESCRIPTION OF THE DRAWINGS

The above, as well as additional objects, features, and advantages of the present invention, will be better understood through the following illustrative and non-limiting detailed description of embodiments of the present disclosure, with reference to the appended drawings, where the same reference numerals will be used for similar elements, wherein:

FIG. 1 shows a system comprising an extracorporeal circuit, a heat transfer system, and a storage device according an embodiment;

FIG. 2 shows a system comprising an extracorporeal circuit, a heat transfer system, and a storage device according to an embodiment;

FIG. 3 shows a flow chart of a medical procedure involving inducing hypothermia in a patient undergoing surgery;

FIG. 4 shows a flow chart of a method for operating a system during a cooling phase and a rewarming phase according to an embodiment;

FIG. 5 shows a flow chart of a method for determining operation mode of a system according to an embodiment; and

FIG. 6 shows a flow chart of a method for determining operation mode of an air-cooled heat exchanger according to an embodiment.

### DETAILED DESCRIPTION

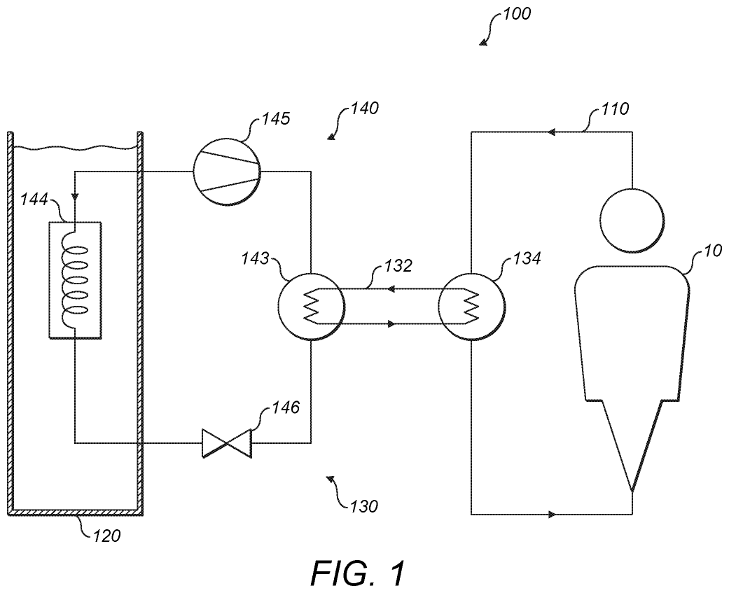

FIG. 1 shows by way of example a system 100 for regulating a body temperature of a patient 10 undergoing surgery, such as, for instance, cardiothoracic surgery. The system 100 may be employed to induce hypothermia in the patient, for example in a Deep Hypothermic Circulatory Arrest (DHCA) procedure.

The system 100 comprises an extracorporeal circuit 110 configured to circulate blood of the patient 10 outside of the patient's body. The system 100 further comprises thermal storage device and a heat transfer system 130 for transferring heat between the blood in the extracorporeal circuit 110 and the storage device 120. The patient 10 may thus be cooled by moving heat from the blood to the storage device 120 and rewarmed by moving heat from the storage device 120 to the blood. In the present example, the heat transfer system 130 is operable to manage the heat transfer in both directions, such that hypothermia may be induced prior the surgical procedure, maintained during the surgery, and the patient 10 be rewarmed after the completion of the surgical procedure.

The heat transfer system 130 comprises a heat transfer circuit 132 configured to circulate a thermal fluid such as water, a first heat exchanger 134 configured to transfer heat between the blood in the extracorporeal circuit 110 and the thermal fluid, as well as a heat pump circuit 140 for transferring heat between the thermal fluid in the heat transfer circuit 132 and the storage device 120. The heat pump circuit 140 is configured to use condensers/evaporators 143, 144 in thermal contact with the thermal fluid and the storage device 120, respectively, a compressor 145, and one or more expansion valves 146 as commonly associated with a thermodynamic refrigeration cycle.

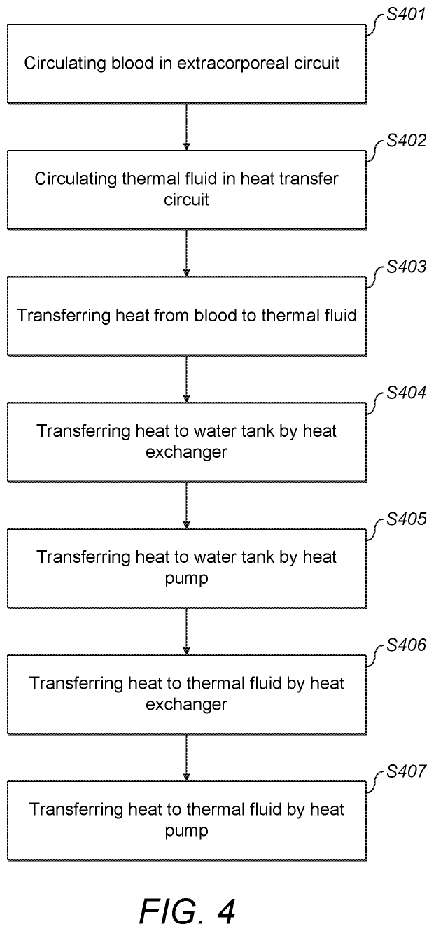

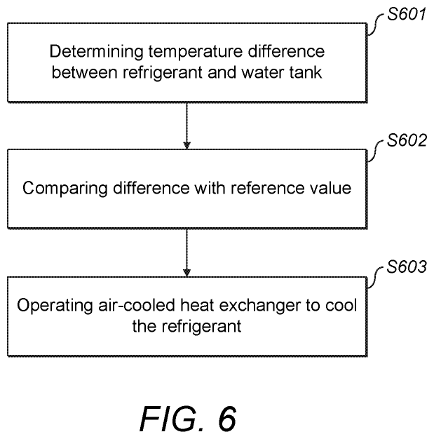

It is appreciated that each of the sub-systems, i.e., the extracorporeal circuit 110, the heat transfer system 130, and the storage device 120 may comprise components for controlling and monitoring the operation of the system 100. These components may be communicatively and operatively connected to one or more control units or processors (not shown), which may perform various operations causing heat to be moved between the patient 10, the storage device 120, and, optionally, the surrounding air. Examples of such operations are disclosed in connection with FIGS. 4-6.

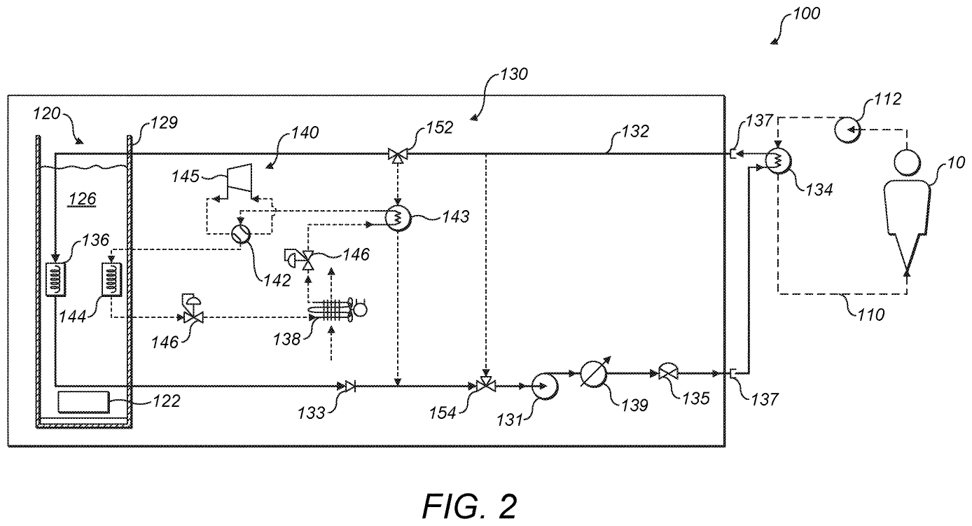

A more detailed example of a system 100, which may be similar to the one in FIG. 1, will now be discussed with reference to FIG. 2.

FIG. 2 shows, by way of example, a system 100 comprising an extracorporeal circuit 110 (indicated by dashed lines), a storage device 120, and a heat transfer system 130 for moving heat between blood in the extracorporeal circuit 110 and the storage device 120 as discussed above in connection with FIG. 1. The extracorporeal circuit 110 comprises a fluid pump 112 arranged to circulate the blood through fluid lines forming the circuit, as well as through the first heat exchanger 134. The first heat exchanger 134 is operable to cool the blood in a cooling phase and heat the blood in a rewarming phase by facilitating heat transfer between the blood circuit 110 and the heat transfer system 130. It will be appreciated that the system illustrated in FIG. 2 is a simplified representation of an actual system and that the extracorporeal circuit 110 may comprise additional components not depicted, such as filters and medical devices for various therapeutic or diagnostic purposes.

The heat transfer circuit 132 (indicated by solid lines), which may be a water circuit, is operable to circulate the thermal fluid through the first heat exchanger 134 during operation of the system 100. Heat can therefore be exchanged between the blood in the extracorporeal circuit 110 and the thermal fluid in the heat transfer circuit 132. The circulation may be controlled by a fluid pump unit 131, which may be controlled by a control unit. Additional components for monitoring and controlling the flow of thermal fluid may also be provided, such as a non-return valve 133 and a flow control valve 135. In some examples, a heater 139 may be provided to heat the thermal fluid. Further, a coupling interface 137 may be provided for releasable engagement between the heat transfer circuit 132 and the first heat exchanger 134.

In some examples, a second heat exchanger 136 is arranged to transfer heat between the thermal fluid and the storage device 120. The second heat exchanger 136 may be arranged in direct thermal contact with the storage device 120 to enable efficient heat exchange. In the example shown in FIG. 2, the second heat exchanger 136 is immersed in the water 126 of the water tank 120. During operation of the second heat exchanger 136, the system 100 may operate in a direct transfer mode in which heat is directly transferred from the heat transfer circuit 132 to the storage device 120. Additionally, or alternatively, the heat pump circuit 140 (indicated by dashed lines) may be arranged to facilitate the transfer of heat between the extracorporeal circuit 110 and the storage device 120. The heat pump circuit 140 may form a thermal link between the heat transfer circuit 132 and the storage device 120 and may thus enable an operating mode referred to as an indirect transfer mode.

In the cooling phase, the evaporator 143 of the heat pump circuit 140 is operated to absorb heat from the thermal fluid in the heat transfer circuit 132, whereas the condenser 144 is arranged to release the heat into the storage device 120. The heat is transferred between the evaporator 143 and the condenser 144 by means of a refrigerant, which is pressurised and pumped through the heat pump circuit 140 by means of the compressor 145. Further, the one or more expansion valves 146 are provided to regulate the flow of refrigerant and ensure it enters the evaporator 143 as a low-pressure, low-temperature liquid.

Similar to the second heat exchanger 136, the condenser 144 is arranged in thermal contact with the storage device 120 to facilitate efficient release of heat from the heat pump circuit 140. In the example shown in FIG. 2, the condenser 144 is immersed in the water tank 120.

As mentioned above, the heat pump circuit 140 may be used in a cooling phase to induce hypothermia in the patient 10 by moving heat from the heat transfer circuit 132 and dump it into the storage device 120. However, in some examples, the heat pump circuit 140 may also be used in a rewarming phase, in which the heat pump circuit 140 is operated in a reverse mode to move heat from the storage device 120 to the heat transfer circuit 132 for further transfer to the extracorporeal circuit 110. In the reverse mode, the evaporator 143 may instead act as a condenser releasing heat to the thermal fluid, whereas the condenser 144 may act as an evaporator absorbing heat from the storage device 120. The reverse mode may be enabled by a reversing valve 142 operable to reverse the flow of refrigerant through the compressor 145.

A valve arrangement 152, such as a three-way flow path selection valve, may be provided to route the flow of thermal fluid through the heat pump circuit 140 only, the second heat exchanger 136 only, or a combination of both. Put differently, the valve arrangement 152 may be operable to at least partly bypass at least one of the heat pump circuit 140 and the second heat exchanger 136. The valve arrangement 152 may hence be used for selecting the operating mode of the system 100, such as changing between the direct transfer mode and the indirect transfer mode.

A mixing valve arrangement 154 may also be arranged to direct a portion of the flow of thermal fluid through the second heat exchanger 136 and/or heat pump circuit 140 and to short-circuit the remaining portion of the flow. By varying a mixing ratio between the short-circuited portion and the cooled/heated portion of the flow, an improved temperature control may be achieved. The bypass is indicated by a dashed line in FIG. 2.

Optionally, an air-cooled heat exchanger 138 may be provided to cool the refrigerant of the heat pump circuit 140. The air-cooled heat exchanger 138 may be arranged in the flow path of the refrigerant and cooled by the surrounding air, for example by means of a fan operable to induce a cooling airflow through the heat exchanger 138.

The storage device 120 may, as indicated in FIGS. 1 and 2, comprise a container, or tank 124, storing water 126. The storage device 120 may, for example, hold about 60 litres of water, which has showed to be sufficient for inducing hypothermia in the patient during surgery. However, the thermal storage capacity of the of the storage device 120 can be increased by a phase change material, such as ice, arranged in thermal contact with the storage device 120. In the present example, a block of ice 122 is immersed in the water tank 120. Beneficially, the phase change material 122 may allow for the size of the storage device to be decreased, thereby increasing mobility and facilitating transporting of the system 100. In the present example, the phase change material 122 allows the water volume to be reduced from about 60 litres to about 30 litres.

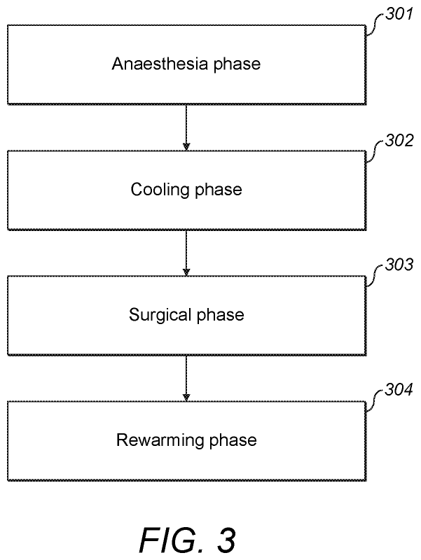

The invention according to the embodiments disclosed in FIGS. 1 and 2 may, in a non-limiting example, be used in a DHCA procedure. FIG. 3 is a schematic representation of various phases of such a procedure.

A first phase may be referred to as the anaesthesia phase 301, in which the patient 10 is anaesthetised, sterile conditions provided, and surgical sites exposed. Further, sensors may be placed for monitoring temperature, blood pressure, oxygen levels, and other vital signs.

In the cooling phase 302, extracorporeal circulation may be initiated via a cardiopulmonary bypass machine, which may take over the function of the heart and lungs during the surgery and allow the blood in the extracorporeal circuit to be cooled. The patient's temperature is gradually lowered to mild or moderate hypothermia, in specific cases to deep hypothermia with a core body temperature below 20° C. Once the desired level of hypothermia is achieved, the surgical phase 303 initiated. In the surgical phase 303, circulatory arrest of the patient's heart may be induced to allow the surgery to be performed under a bloodless and motionless field.

Once the surgical phase 303 is complete, the patient's body temperature may be gradually returned to normal physiological levels during a rewarming phase 304.

The system 100 disclosed herein may be used to regulate the temperature of a patient undergoing surgery, for example involving a DHCA procedure as outlined in FIG. 3. An example of how such a system 100 can be used in a cooling phase 302 and in a rewarming phase 304 will now be described with reference to FIG. 4.

In the cooling phase 302, heat is removed from the extracorporeal circuit 110 and absorbed by the storage device 120 (in the following exemplified by a water tank 120). The heat transfer may be performed by fluidically separated circuits, such as the extracorporeal circuit 110, the heat transfer circuit 132, and the heat pump circuit 140. In a typical operation of the system 100, a first pump unit 112 is operated to circulate S401 blood in the extracorporeal circuit 110, whereas a second pump unit 131 is operated to circulate S402 the thermal fluid in the heat transfer circuit 132. Heat may be transferred S403 from the blood to the thermal fluid by means of the first heat exchanger 134 and transferred from the thermal fluid to the storage device 120 either by a second heat exchanger 136, or by the heat pump circuit 140. In an initial phase, the heat may be transferred S404 by the second heat exchanger 136. In a subsequent phase, the heat may be transferred S405 by the heat pump circuit.

Typically, it may be more efficient to use the second heat exchanger 136 during the initial phase of the cooling phase, when there is a relatively large temperature difference between the storage device and the patient and use the heat pump circuit 140 for smaller temperature differences and when the temperature in the water tank 120 exceeds the temperature of the patient 10. The cooling may hence be divided into an initial phase in which the heat transfer system 130 is operated in the direct transfer mode to move heat directly to the storage device 120, and a subsequent phase in which the heat transfer system 130 is operated in the indirect transfer mode to move heat to the storage device 120 indirectly via the heat pump circuit 140.

In an example, the patient may be approximated by a volume of 80 litres of water that needs to be cooled from 37° C. to 20° C. To achieve this, the system is initially operated in the direct transfer mode for about 10 minutes to reduce the body temperature by 10-15° C. while the temperature of the water tank increases from 5° C. to 18° C. Thereafter, the system is switched to the indirect transfer mode for about 40 minutes to further reduce the body temperature to 20° C. while increasing the temperature of the water tank to 43° C. by means of the heat pump circuit 140. Thereafter, a constant temperature phase is entered, in which the refrigerant of the heat pump circuit 140 is cooled by the air-cooled heat exchanger 138 until the rewarming phase 304 is initiated.

If the patient is approximated by a body of 80 litres of water, about 5,700 kJ needs to be removed to reduce the temperature to 20° C. Transferring 5,700 kJ in 50 minutes, as mentioned above, would correspond to an average cooling power of about 1.9 kW. This can be compared with the constant temperature phase, which can be maintained by dissipating as little as about 200-300 W from the heat pump circuit 140. As the cooling power required to maintain the constant temperature phase is relatively small, this can be achieved by an air-cooled heat exchanger 138 without significantly affecting the environment in the operating theatre.

In the rewarming phase 304, the heat transfer system 130 may be operated to remove heat from the storage device 120 and transfer it to the extracorporeal circuit 110. Initially, the second heat exchanger 136 may be operated to transfer S406 heat from the storage device 120 to the thermal fluid in the heat transfer circuit 132. In a subsequent phase, the heat pump circuit 140 may be operated in reversed mode to transfer S407 heat from the storage device to the thermal fluid. In the above example, the initial warming phase lasts for about 10 minutes until the temperature of the water tank reaches 37° C. Thereafter, the heat pump circuit 140 is used until the normal body temperature of the patient is reached. At this point, the temperature of the water tank is about 20° C.

The selection of operation mode of the system 100 may hence be based on a temperature difference between the body temperature of the patient 10 and the temperature of the storage device 120. A controller or one or more processors of the system may be arranged to receive sensor data indicative of a core body temperature of the patient 10 as well as of the storage device 120, and to control the system accordingly.

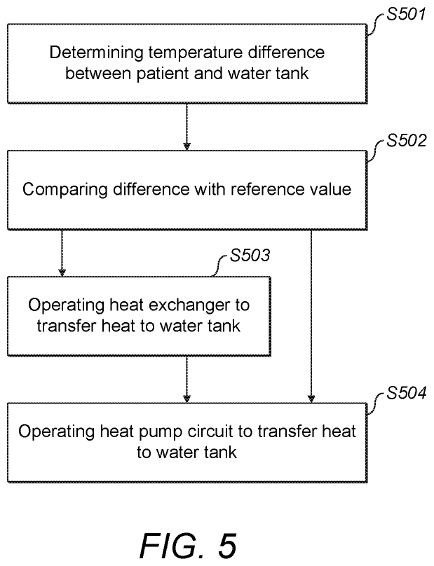

In an example, illustrated in FIG. 5, the system 100 may be configured to determine S501 the difference between the body temperature of the patient and a temperature of the storage device 120. The body temperature of the patient may be retrieved from sensor data provided by monitoring equipment monitoring vital parameters of the patient during the anaesthesia, whereas the storage device temperature may be retrieved from sensor data delivered by a thermometer measuring the water temperature in the water tank 120. The difference between the two temperatures may then be compared S502 with a reference value, such as a predetermined threshold value, which may indicate a limit below which the heat transfer efficiency of the second heat exchanger 136 is too low for practical purposes.

If the temperature difference meets or exceeds the threshold value, the second heat exchanger 136 may be operated S503 to transfer the heat from the thermal fluid to the storage device 120. If the temperature difference is below the threshold value, the heat pump circuit may be operated S504 to transfer the heat from the thermal fluid to the storage device 120. The controller may be configured to cause the flow path selection valve 152 to bypass the heat pump circuit 140 in response to the temperature difference meeting or exceeding the threshold value, and to cause the flow path selection valve 152 to bypass the second heat exchanger 136 in response to the temperature difference being below the threshold value.

The selection of operation mode based on the difference between the patient's body temperature and the temperature of the storage tank 120 may be applied both during the cooling phase 302 and the rewarming phase 304.

A similar approach may be used for operating the air-cooled heat exchanger 138, which may be arranged to dissipate heat from the refrigerant of the heat pump circuit 140. The operation of the air-cooled heat exchanger 138 may involve determining S601 a temperature difference between the refrigerant of the heat pump circuit and the storage device 120, comparing S602 the temperature difference with a predetermined threshold value, and operating S603 the air-cooled heat exchanger 138 to cool the refrigerant based at least in part on the temperature difference being below the threshold value. The threshold value may be selected to indicate a temperature difference below which the heat transfer efficiency of the refrigeration cycle is too low to be of any practical use. Put differently, the air-cooled heat exchanger 138 may be employed to dissipate heat from the system 100 in cases when the storage unit 120 is approaching its maximum storage capacity. In alternative examples, the air-cooled heat exchanger 138 is activated to maintain a steady-state temperature during, for example, the surgical phase 303, in which it is operated to dissipate heat generated by the patient's body and medical equipment. Further, it may be used to reduce the temperature of the storage device 110 during a regeneration phase in which the system 100 is prepared for a new patient 10.

The system may generally comprise one or more processors and one or more non-transitory computer-readable media storing first computer executable instructions that, when executed by the one or more processors, cause the system to perform at least parts of the actions shown in FIGS. 4-6 and described above.

Generally, the system 100 may comprise circuitry which is configured to implement (using one or more non-transitory computer-readable media) the functionality described herein. Suitable processors for the execution of a program of instructions include, by way of example, both general and special purpose microprocessors, and the sole processor or one of multiple processors or cores, of any kind of computer. The processors can be supplemented by, or incorporated in, ASICs (application-specific integrated circuits). Those skilled in the art will understand that the above-described exemplary embodiments may be implemented in any suitable software, hardware, or firmware configuration or combination thereof. An exemplary hardware platform for implementing the exemplary embodiments may include, for example, an Intel x86 based platform with compatible operating system, a Windows OS, a Mac platform and MAC OS, a mobile device having an operating system such as iOS, Android, etc. In a further example, the exemplary embodiments of the above-described method may be embodied as a program containing lines of code stored on a non-transitory computer readable storage medium that, when compiled, may be executed on a processor or microprocessor.

Additionally, variations to the disclosed embodiments can be understood and effected by the skilled person in practising the claimed invention, from a study of the drawings, the disclosure, and the appended claims. Moreover, in the drawings and specification, there have been disclosed preferred embodiments and examples of the invention and, although specific terms are employed, they are used in a generic and descriptive sense only and not for the purpose of limitation. The scope of the invention is set forth in the following claims, in which the word ‘comprising’ does not exclude other elements or steps, and the indefinite article ‘a’ or ‘an’ does not exclude a plurality.

1. A system for regulating a body temperature of a patient undergoing surgery, the system comprising:
an extracorporeal circuit configured to circulate blood of the patient outside a body of the patient;
a storage device configured to accumulate thermal energy; and
a heat transfer system configured to transfer heat between the blood in the extracorporeal circuit and the storage device;
wherein the heat transfer system comprises:
a heat transfer circuit configured to circulate a thermal fluid;
a first heat exchanger configured to transfer heat between the blood in the extracorporeal circuit and the thermal fluid; and
a heat pump circuit configured to transfer heat between the thermal fluid and the storage device.

2. The system according to claim 1, wherein the heat transfer system further comprises a second heat exchanger configured to transfer heat between the thermal fluid and the storage device.

3. The system according to claim 2, further comprising a valve arrangement configured to allow the thermal fluid to at least partly bypass at least one of the heat pump circuit and the second heat exchanger.

4. The system according to claim 1, wherein the heat transfer system further comprises an air-cooled heat exchanger configured to cool a refrigerant of the heat pump circuit.

5. The system according to claim 1, wherein the heat transfer circuit further comprises a heater configured to heat the thermal fluid.

6. The system according to claim 1, wherein the heat pump circuit comprises a reverse valve arrangement configured to control a direction of operation of the heat pump circuit, such that the heat pump circuit is selectively operable in a cooling mode in which heat is transferred from the thermal fluid to the storage device, and in a heating mode in which heat is transferred from the storage device to the thermal fluid.

7. The system according to claim 1, wherein the storage device further comprises a phase-change material configured to absorb or release heat from/to the thermal fluid.

8. The system according to claim 1, wherein the storage device is a liquid reservoir.

9. The system according to claim 1, wherein the storage device is a container configured to accommodate water.

10. The system according to claim 1, wherein the thermal fluid is water.

11. One or more non-transitory computer-readable media storing instructions executable by one or more processors, wherein the instructions, when executed, cause the one or more processors to perform operations comprising:
operating a first pump unit to circulate blood of a patient in an extracorporeal circuit;
operating a second pump unit to circulate a thermal fluid in a heat transfer circuit, wherein the blood in the extracorporeal circuit and the thermal fluid in the heat transfer circuit are in thermal contact with each other to allow transfer of heat between the blood and the thermal fluid; and
operating a heat transfer system to transfer heat between the thermal fluid in the heat transfer circuit and a storage device configured to accumulate thermal energy.

12. The one or more non-transitory computer-readable media according to claim 11, wherein the operations comprise at least one of:
operating a heat exchanger to transfer the heat between the thermal fluid and the storage device; and
operating a heat pump circuit to transfer the heat between the thermal fluid and the storage device.

13. The one of more non-transitory computer-readable media according to claim 12, wherein the operations comprise:
determining a temperature difference between a body temperature of the patient and a temperature of the storage device;
comparing the temperature difference with a predetermined threshold value;
operating the heat exchanger to transfer the heat from the thermal fluid to the storage device based at least in part on the temperature difference meeting or exceeding the threshold value; and
operating the heat pump circuit to transfer the heat from the thermal fluid to the storage device based at least in part on the temperature difference being below the threshold value.

14. The one or more non-transitory computer-readable media according to claim 12, wherein the operations comprise:
determining a temperature difference between a refrigerant of the heat pump circuit and a temperature of the storage device;
comparing the temperature difference with a predetermined threshold value; and
operating an air-cooled heat exchanger to cool the refrigerant based at least in part on the temperature difference being below the threshold value.

15. A method for regulating a body temperature of a patient undergoing surgery, comprising:
circulating blood of the patient in an extracorporeal circuit;
circulating a thermal fluid in a heat transfer circuit;
transferring heat between the blood in the extracorporeal circuit and the thermal fluid in the heat transfer circuit; and
transferring heat between the thermal fluid and a storage device configured to accumulate thermal energy.

16. The method according to claim 15, wherein the transferring the heat between the thermal fluid and the storage device includes using at least one of a heat exchanger and a heat pump.
